(19) United States
(12) Patent Application Publication
(10) Pub. No.: US 2007/0162088 A1
(43) Pub. Date: Jul. 12, 2007

(54) Apparatus for stimulating muscular contraction and relaxation

(76) Inventors: Chiang-Chuan Chen, Taipei City (TW); Rai-Chi Chan, Taipei City (TW)

Correspondence Address: BIRCH STEWART KOLASCH & BIRCH, PO BOX 747, FALLS CHURCH, VA, 22040-0747, US

(21) Appl. No.: 11/395,184

(22) Filed: Apr. 3, 2006

(30) Foreign Application Priority Data
Jan. 12, 2006 (TW) 095200713

(51) Int. Cl. A61N 1/36

(52) U.S. Cl. 607/48

(57) ABSTRACT

An apparatus for stimulating muscular contraction and relaxation for a human body includes a specific needle and a host. The specific needle, made of conductive metal, is coated with some insulating material on an outer layer thereof, such that a tip of the needle is exposed outside the insulating material. The specific needle is electrically coupled to a signal cable. A tip of the specific needle, with a surface thereof not coated with the insulating material, is exposed outside the insulating material. The host is coupled to the specific needle via the signal cable. The specific needle pierces into a target point of the muscle and transmits a control signal to the specific needle via the signal cable in order to lead a micro-current into the target point of the muscle, thereby making the muscle contract and relax.

### BACKGROUND OF THE INVENTION

(a) Field of the Invention

The present invention relates to an apparatus for stimulating muscular contraction and relaxation, more particularly to an apparatus for stimulating muscular contraction and relaxation by stimulating trigger points via electric current.

(b) Description of the Prior Art

Nowadays, a faster pace of life and a reduced activity level cause a lot of muscular illnesses. Among them, myofascial pain becomes a common muscular illness. According to statistics, the prevalence of myofascial pain is rather high and is approximately 14.4% of the world's population. The symptoms of myofascial pain include muscular tension, aching, sense of feebleness, and the development of trigger points, taut bands, and referred pain when muscles are touched. Myofascial pain is not fatal, but is rather annoying, easily causes chronic illnesses, and affects the quality of life of the patient.

The existing treatment for myofascial pain primarily includes: drug therapy, rehabilitation, and improvement in one's living styles. For drug therapy, non-steroidal anti-inflammatory drugs (NSAIDs), muscle relaxants, and so on are used. Rehabilitation therapy includes hot pack, interferential therapy, hydrotherapy, stretching, relaxation, kinesio taping, and biofeedback training. Improvement in one's life styles includes regular daily living, regular exercises, and relaxation.

Despite this, long-term drug therapy easily induces side effects. The time required for rehabilitation therapy is rather long, and its effects differ greatly with people. The effects on the improvement in one's life styles are to be enhanced. Therefore, it is necessary to develop an apparatus for alleviating muscular tension in order to reduce the incidence of myofascial pain.

### SUMMARY OF THE INVENTION

The primary object of the present invention is to provide an apparatus for stimulating muscular contraction and relaxation, by leading a micro-current to specific trigger points of muscles in order to promote muscular contraction and relaxation and relieve muscular tension.

To achieve the above-mentioned object and other objects, the present invention provides an apparatus for stimulating muscular contraction and relaxation, for use with a human body. The apparatus includes a specific needle and a host. The specific needle is made of conductive metal. An outer layer of the specific needle is coated with some insulating material such that a tip of the needle is exposed outside the insulating material. The specific needle is electrically coupled to a signal cable. The tip of the needle, with a surface thereof not coated with the insulating material, is exposed outside the insulating material. Moreover, the host is coupled to the specific needle via the signal cable. The specific needle pierces into a target point of the muscle at a depth of D cm. D is a real number. A control signal is transmitted to the specific needle via the signal cable in order to lead a micro-current into the target point of the muscle, thereby making the muscle contract and relax.

According to a preferred embodiment of the present invention, the host comprises an operating unit, a driver unit, a display unit, and a control unit, wherein the operating unit adjusts the control signal and the driver unit outputs a micro-current. The control unit is coupled to the operating unit, the driver unit, and the display unit.

According to another preferred embodiment of the present invention, the target point is a trigger point.

According to another preferred embodiment of the present invention, the control signal is a direct current (DC) pulse.

According to another preferred embodiment of the present invention, the frequency of the DC pulse lies between 2 Hz and 200 Hz.

According to another preferred embodiment of the present invention, the width of the DC pulse lies between 400 μsec and 1400 μsec.

According to another preferred embodiment of the present invention, the micro-current is a constant current at 1 milli-ampere (mA) to 30 milli-amperes (mA).

According to another preferred embodiment of the present invention, the control signal acts for 2 minutes to 5 minutes and then auto stops.

According to another preferred embodiment of the present invention, a head of the specific needle is conical in shape.

According to another preferred embodiment of the present invention, an area of 0.2 mm² to 0.4 mm² is coated with the insulating material at an end of the specific needle.

In summary, the present invention provides an apparatus for stimulating muscular contraction and relaxation. After stimulating muscular contraction and relaxation of a myofascial pain-affected patient by the apparatus of the present invention, regular electrical stimulation can contract and relax muscles, thereby restoring the tension of the normal muscular tissues outside the target points such that convulsions no longer happen to the muscles. Moreover, the stimulation of muscles can cause localized muscular edema and the removal of metabolites, such that metabolites such as water, creatinine, and calcium originally accumulated inside the muscles are removed, thereby reducing the stimulation on pain nerves and the nuclear bags and nuclear chain fibers that cause reflex convulsions. In addition, the repeated constant-frequency contraction of the target points can deplete the release of excitatory acetylcholine on neuromuscular junctions, thereby reducing the contraction intensity and convulsion intensity of muscular cells. As muscles at convulsions rapidly relax, it is possible to speed up the reparation of injured or inflamed tissues. On the one hand, the structure of the tissues at the target points is like that of the damaged neuromuscular junctions. In this way, the micro-current can enhance cellular regeneration, thereby restoring abnormal tissues to normal functions. On the other hand, the micro-current can sequentially ablate the fibrous adhesive tissues that have suffered from long-term pain and allow the regeneration of normal muscular tissues. In addition, non-active repeated muscular contractions drive the contraction and relaxation of the smooth muscles in the dermis and increase the micro-circulation of the epidermis on the treated parts.

To enable a further understanding of the objectives and the technological methods of the invention herein, the brief description of the drawings below is followed by the detailed description of the preferred embodiments.

### BRIEF DESCIPTION OF THE DRAWINGS

FIG. 1 shows a schematic view of the structure of an apparatus for stimulating muscular contraction and relaxation according to a preferred embodiment of the present invention.

FIG. 2 shows a schematic view of the structural of a specific needle according to a preferred embodiment of the present invention.

FIG. 3 shows a view of an apparatus for stimulating muscular contraction and relaxation according to another preferred embodiment of the present invention.

FIG. 4 shows schematic view when the specific needle pierces into a target point of the muscular according to another preferred embodiment of the present invention.

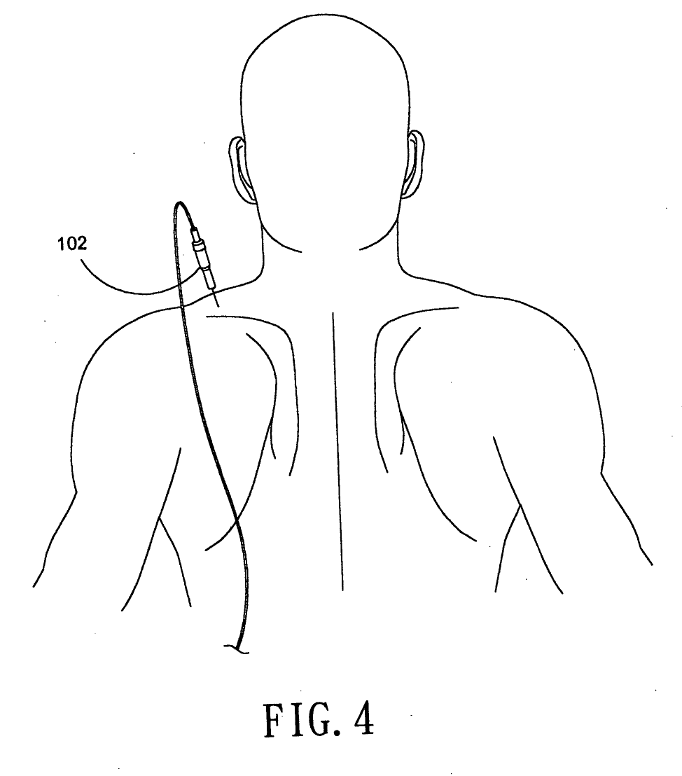

### DETAILED DESCRIPTION OF THE PREFERRED EMBODIMENTS

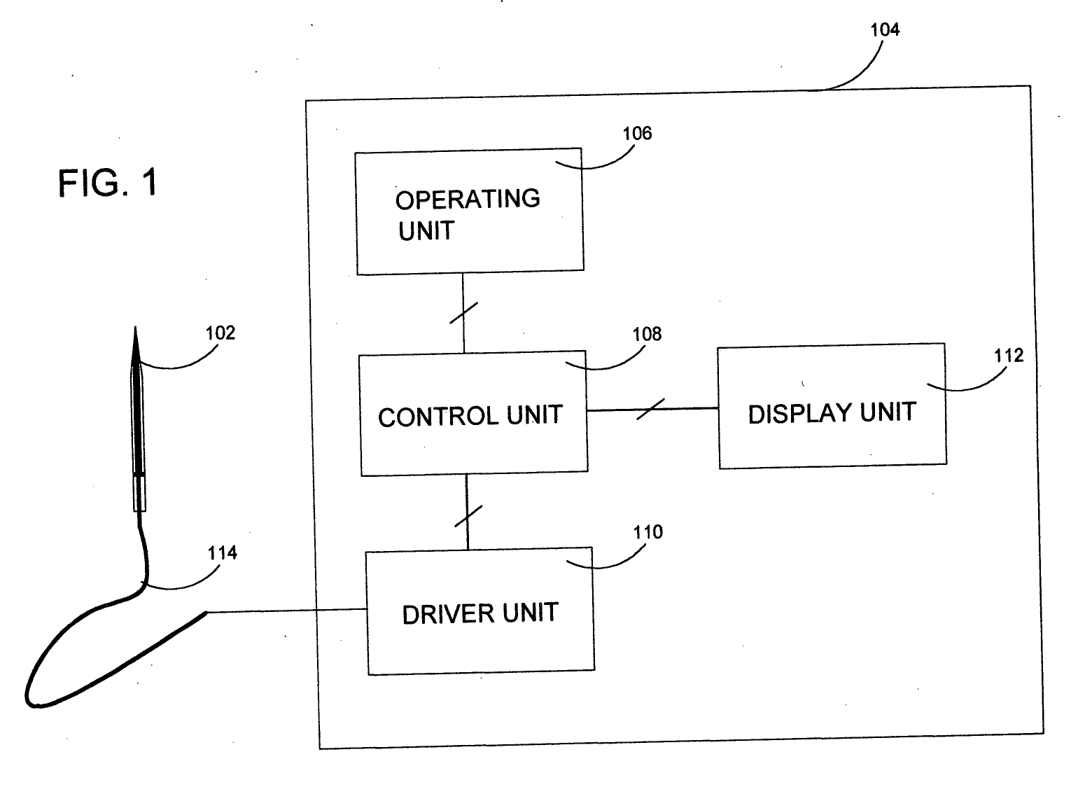

Referring to FIG. 1, an apparatus for stimulating muscular contraction and relaxation 100 includes a needle body 102 and a host 104, wherein the host 104 includes an operating unit 106, a control unit 108, a driver unit 110, and a display unit 112, wherein the needle body 102 is electrically coupled to the host 104 via a signal cable 114, and the control unit 108 is coupled to the operating unit 106, the driver unit 110, and the display unit 112.

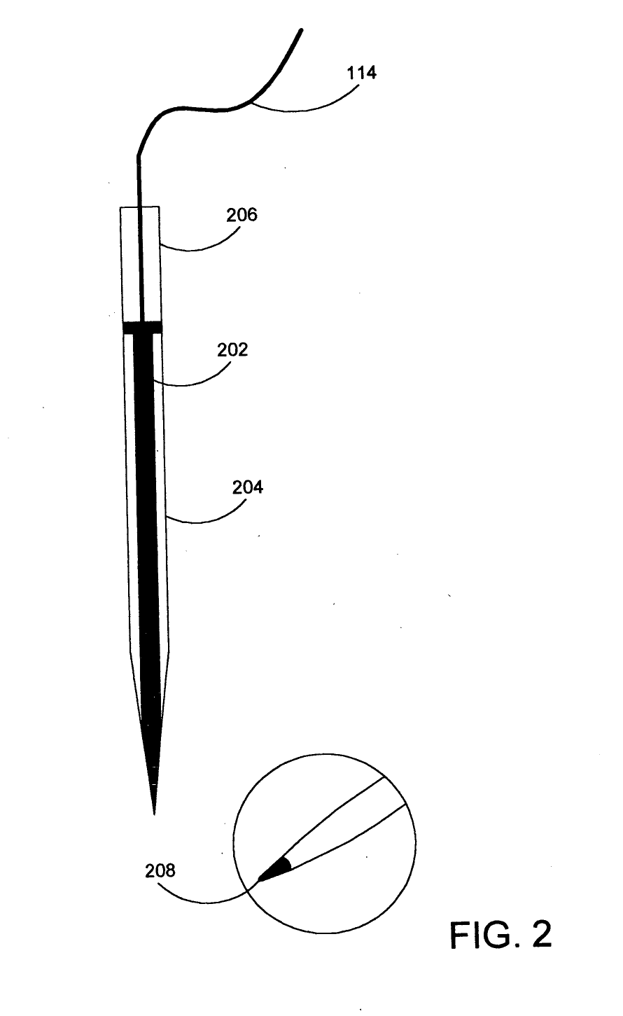

Referring to FIG. 2, the needle body 102 includes a specific needle 202, an insulating material 204, and a needle stem 206, wherein the specific needle 202 is made of conductive metal, having a head thereof conical in shape, and is electrically coupled to the signal cable 114. An outer layer of the specific needle 202 is coated with some insulating material 204 such that a tip of the needle 202 is exposed outside the insulating material 204. Moreover, the tip of the needle 202, with a surface thereof not coated with the insulating material 204, is exposed outside the insulating material 204. The tip of the specific needle 202 extends for an area of 0.2 mm² to 0.4 mm² from a top of the insulating material 204.

When operating the apparatus for stimulating muscular contraction and relaxation 100, the needle body 102 pierces into a target point of the human muscle at a depth of D cm. D is a real number. A control signal is transmitted to the specific needle 202 via the signal cable 114 in order to lead the micro-current into the target point of the muscle, thereby making the muscle contract and relax. After the specific needle 202 has pierced into the muscle, the sensory nerves on a shallow layer of the skin comes into contact with the insulating material 204, and the tip of the specific needle 202 comes into direct contact with the target point. Therefore, when the micro-current is led into the target point of the muscle, the human body will no longer feel aching or paralyzed.

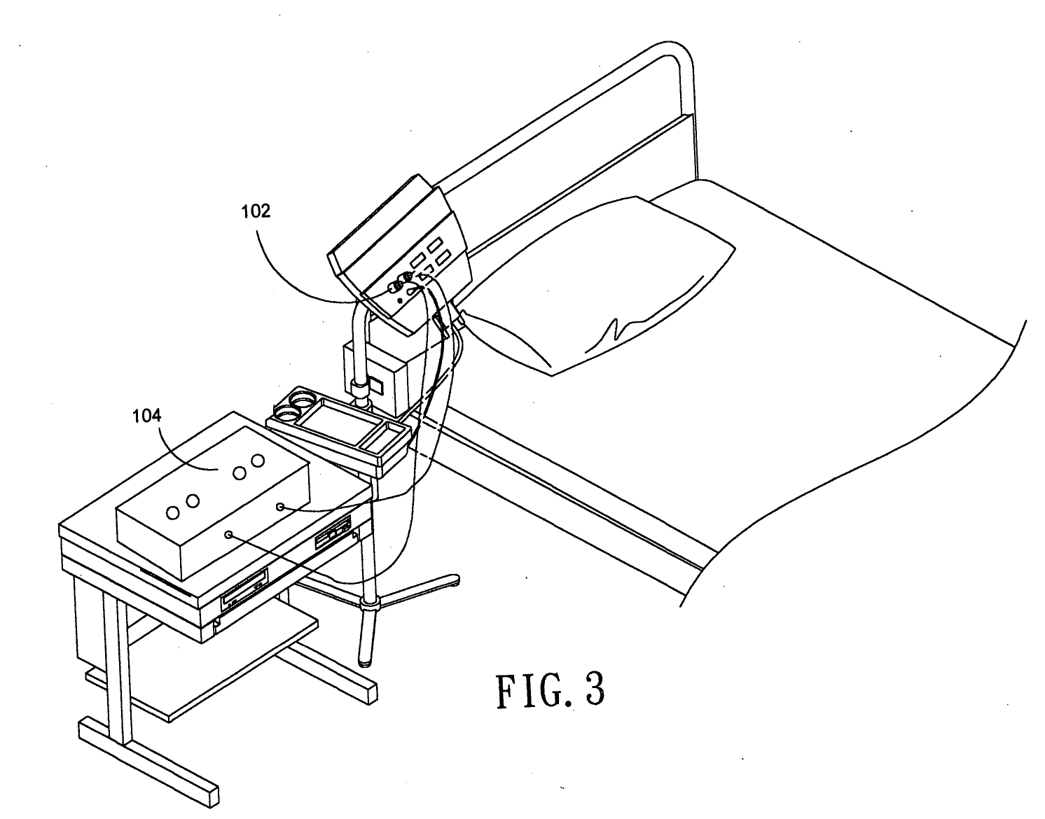

Referring to FIG. 3, the target point is, medically speaking, the trigger point. Moreover, the control signal is the DC pulse. According to the optimum results of clinical experiments, the frequency of the DC pulse lies between 2 Hz and 200 Hz; the width of the DC pulse lies between 400 μsec and 1400 μsec; the micro-current is a constant current at 1 milli-ampere (mA) to 30 milliamperes (mA). The control signal acts for 2 minutes to 5 minutes and then auto stops. The medical mechanism of the apparatus for stimulating muscular contraction and relaxation 100 on the human body is to relax the taut band at the deep layers of the muscles, promote blood circulation, block the stimulation on the neuromuscular junctions, and damage the localized micro-adhesive tissues. Referring to FIG. 3, which shows schematic view when the specific needle pierces into a target point of the muscular according to another preferred embodiment of the present invention.

According to a clinical experiment, the apparatus for stimulating muscular contraction and relaxation 100 was applied on 870 myofascial pain-affected patients for muscular contraction and relaxation. According to the statistical analysis of the results, if the rating of pain perception was set to be 10 points, straight after applying the stimulation, 83.15% of patients immediately perceived an improvement whereas 16.85% of patients perceived no improvement. Among those indicating an improvement in pain perception, 31.54% of them perceived an improvement of one point; 26.16% of them perceived an improvement of two points; 25.45% of them perceived an improvement of three points or above. After applying the stimulation for one week, 80.25% of patients perceived an improvement whereas 19.75% of patients perceived no improvement. Among those indicating an improvement in pain perception, 28.4% of them perceived an improvement of one point; 16.05% of them perceived an improvement of two points; 35.8% of them perceived an improvement of three points or above.

After applying the apparatus for stimulating muscular contraction and relaxation 100 on the myofascial pain-affected patients for muscular contraction and relaxation, Nuclear Magnetic Resonance (NMR) testing results of the brain show that the pain-control region of the patient's brain (that is around the cerebral aqueduct) has an enhanced activity, suggesting that after stimulating muscular contraction and relaxation, this can affect the pain activities of the brain center, thereby proving that the apparatus for stimulating muscular contraction and relaxation 100 can relax muscles and has central pain inhibitory actions.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. An apparatus for stimulating muscular contraction and relaxation for a human body, comprising:
a specific needle, made of conductive metal, electrically coupled to a signal cable and coated with some insulating material on an outer layer thereof, wherein a tip of said needle is exposed outside said insulating material; and
a host, coupled to said specific needle via said signal cable; piercing said specific needle into a target point of said muscle at a depth of D cm, with D as a real number, and transmitting a control signal to said specific needle via said signal cable in order to lead a micro-current into said target point of said muscle, thereby making said muscle contract and relax.

2. The apparatus for stimulating muscular contraction and relaxation as claimed in claim 1, wherein said host comprises:
an operating unit, for adjusting said control signal;
a driver unit, outputting said micro-current;
a display unit; and
a control unit, coupled to said operating unit, said driver unit, and said display unit.

3. The apparatus for stimulating muscular contraction and relaxation as claimed in claim 1, wherein said target point is a trigger point.

4. The apparatus for stimulating muscular contraction and relaxation as claimed in claim 1, wherein said control signal is a direct current (DC) pulse.

5. The apparatus for stimulating muscular contraction and relaxation as claimed in claim 4, wherein the frequency of said DC pulse lies between 2 Hz and 200 Hz.

6. The apparatus for stimulating muscular contraction and relaxation as claimed in claim 4, wherein the width of said DC pulse lies between 400 μsec and 1400 μsec.

7. The apparatus for stimulating muscular contraction and relaxation as claimed in claim 1, wherein said micro-current is a constant current at 1 milli-ampere (mA) to 30 milli-amperes (mA)

8. The apparatus for stimulating muscular contraction and relaxation as claimed in claim 1, wherein said control signal acts for 2 minutes to 5 minutes and then automatically stops.

9. The apparatus for stimulating muscular contraction and relaxation as claimed in claim 1, wherein a head of said specific needle is conical in shape.

10. The apparatus for stimulating muscular contraction and relaxation as claimed in claim 1, wherein a tip of said specific needle exposes an area of 0.2 mm² to 0.4 mm² outside said insulating material.
